(19) United States
(12) Patent Application Publication
(10) Pub. No.: US 2003/0114716 A1
(43) Pub. Date: Jun. 19, 2003

(54) Method of producing bisphenol-A

(76) Inventor: Barrie W. Jackson, Perth (CA)

Correspondence Address: DOWELL & DOWELL PC, SUITE 309, 1215 JEFFERSON DAVIS HIGHWAY, ARLINGTON, VA, 22202

(21) Appl. No.: 10/231,186

(22) Filed: Aug. 30, 2002

Related U.S. Application Data
(60) Provisional application No. 60/315,973, filed on Aug. 31, 2001.

(51) Int. Cl. C07C 37/20

(52) U.S. Cl. 568/728

(57) ABSTRACT

Acetone and phenol are reacted over a strong acid catalyst in the presence of lower alcohol. The lower alcohol can be methanol, ethanol, n-propanol, iso-propanol, n-butanol, sec-butanol, tertiary-butanol and iso-butanol. For each mole of acetone one mole or more of lower alcohol is provided, and one to four moles of lower alcohol are preferred.

### CROSS-REFERENCE TO RELATED APPLICATION

This application claims the benefit of the filing date of U.S. Provisional Patent Application No. 60/315,973 filed Aug. 31, 2001 by applicant Barrie W. Jackson entitled Method of Producing Bisphenol-A (BPA). This is the specific reference to the provisional application that is required under 35 U.S.C. 119(e).

### FIELD OF THE INVENTION

The invention relates to the production of Bisphenol-A (BPA). More particularly it relates to the production of BPA using acetone and phenol as reactants.

### BACKGROUND OF THE INVENTION

4,4′-isopropylidenediphenol, more commonly known by its commercial name BPA, is a chemical that is a valuable raw material in the production of many polymers, and is a fundamental building block for epoxy resins and polycarbonates. Its end products are used for many applications, including: adhesives, potting compounds, clear protective cases, headlight covers, and lenses. The use of BPA is growing rapidly in response to the demand for products such as compact discs and digital video discs.

BPA is conventionally formed from the acid-catalyzed reaction of acetone and phenol. In the presence of a strong acid catalyst, yields of 95% of the preferred 4,4′-isopropylidenediphenol (para, para-isomer or BPA) can be achieved while the other 5% consists of 2,4′-isopropylidenediphenol (ortho, para-isomer), and 2,2′-isopropylidenediphenol (ortho, ortho-isomer), which does not occur in any significant amount, and various by-products. BPA is thermodynamically favored in the reaction. The presence of up to 5% of unusable isomers in the reaction product requires a significant number of downstream purification steps in order to achieve the 99.9% BPA purity levels required in the making of polycarbonates for optical discs and similar applications. Otherwise, the colour and mechanical properties of the polymer are impaired. Purity determines whether or not it is cost efficient for manufacturers to produce BPA.

In addition to the isomer distribution the manufacturer will encounter various refractory by-products in the reaction product, the presence of which render the extraction of BPA, as well as the recyclable reactants, costly. Many of these by-products are the consequence of acetone aldol chemistry occurring simultaneously in the reactor with the BPA reaction. Mesityl oxide, for example, resulting from aldol chemistry, is a very reactive compound, and it would be expected to react with phenol to produce by-products such as chromans. Many other compounds resulting from phenol reacting with acetone aldol condensation products are also likely. Among these byproducts are triphenol II, chroman I and II, spirobiindan, 2-(p-hydroxyphenyl)-4-methlypent-3-en-2-ol (C1), and 4-(p-hydroxyphenyl)-2-methyl-1,3-pentadiene (C2). Of these, triphenol II, chroman I, chroman II, C1, and C2 are adducts of phenol and mesityl oxide. Acetone aldol chemistry can also lead to the production of phorone. Spirobiindan is a 2:1 adduct of phenol and phorone.

Based on work previously done under the direction of the inventor (see project description at http://members-http-4.rwcl.sfba.home.net/dchoen/QUFYAS/), it was hypothesized that substitution of dimethoxypropane (DMP) for acetone might decrease the production of by-products. DMP is a recognized acetone substitute, and by substituting it for the acetone it was hoped that the acetone-aldol reactions would be avoided entirely. It was found that while this substitution improved the isomer ratio in favour of the para,para-isomer (BPA), it did not affect by-product production. It was recognized that DMP is not an economically viable substitute for acetone.

It is an object of the invention to address these or other problems associated with the production of BPA.

### SUMMARY OF THE INVENTION

In a first aspect the invention provides a method of making BPA by reacting acetone and phenol over a strong acid catalyst in the presence of at least one lower alcohol selected from methanol, ethanol, n-propanol, iso-propanol, n-butanol, sec-butanol, tertiary-butanol and iso-butanol. In one embodiment, there is at least one mole of lower alcohol for each mole of acetone. In another embodiment, there is at least two moles of lower alcohol for each mole of acetone. In another embodiment, there is no more than four moles of lower alcohol for each mole of acetone. In a preferred embodiment, there is at least one mole of phenol for each mole of acetone prior to reacting the acetone and phenol.

In various embodiments, the lower alcohol is at least one alcohol selected from the group consisting of methanol, ethanol, propanol and butanol. The lower alcohol may be isopropyl alcohol. The lower alcohol may be tertiary butyl alcohol. In a preferred embodiment, the lower alcohol is methanol. The acetone and phenol may be reacted at less than about 120° C. The acetone and phenol may be reacted at greater than about 55° C. The acetone and phenol may be reacted at greater than about 70° C. The catalyst may be an acid cationic resin. In a more preferred embodiment, the invention provides a method of making BPA by reacting acetone and phenol over a strong acid catalyst in the presence of methanol, wherein at least one mole of methanol is present for each mole of acetone.

In a third aspect the invention provides a method of making BPA by adding a lower alcohol selected from methanol, ethanol, n-propanol, iso-propanol, n-butanol, sec-butanol, tertiary-butanol and iso-butanol to a condensation unit of an acetone-phenol BPA production process, wherein at least one mole of the lower alcohol is present for each mole of acetone in the condensation unit. The methods of the invention can be implemented in various systems and processes.

### BRIEF DESCRIPTION OF THE DRAWINGS

For a better understanding of the present invention and to show more clearly how it may be carried into effect, reference will now be made, by way of example, to the accompanying drawings which show the preferred embodiment of the present invention and in which:

FIG. 1 is a block diagram of a modified BPA production process according to a preferred embodiment of the present invention,

FIG. 2 is a chart showing experimental BPA Yield (wt %) with varying Phenol:Acetone:Methanol ratio,

FIG. 3 is a chart showing the occurrence of by-products during BPA production in the experiment of FIG. 2, and

FIG. 4 is a chart showing the number of by-products within +/−5 min of BPA elution time during mass spectrometry analysis of the products of the experiment of FIG. 2.

### DETAILED DESCRIPTION OF THE PREFERRED EMBODIMENT

The present invention is based, in part, on the presence of a lower alcohol in the reaction of phenol and acetone for the production of BPA substantially improving the purity of the BPA, i.e., the number and quantity of by-products formed are reduced. In accordance with a broad aspect of the invention, at least one mole of lower alcohol is present for each mole of acetone in the acetone-phenol condensation production of BPA. It is preferable to have present from two moles to four moles of lower alcohol for each mole of acetone. The amount of phenol used is the same as that used for a given amount of acetone in a conventional acetone-phenol BPA production process, usually greater than one mole of phenol for each mole of acetone and preferably between two moles and four moles of phenol for each mole of acetone.

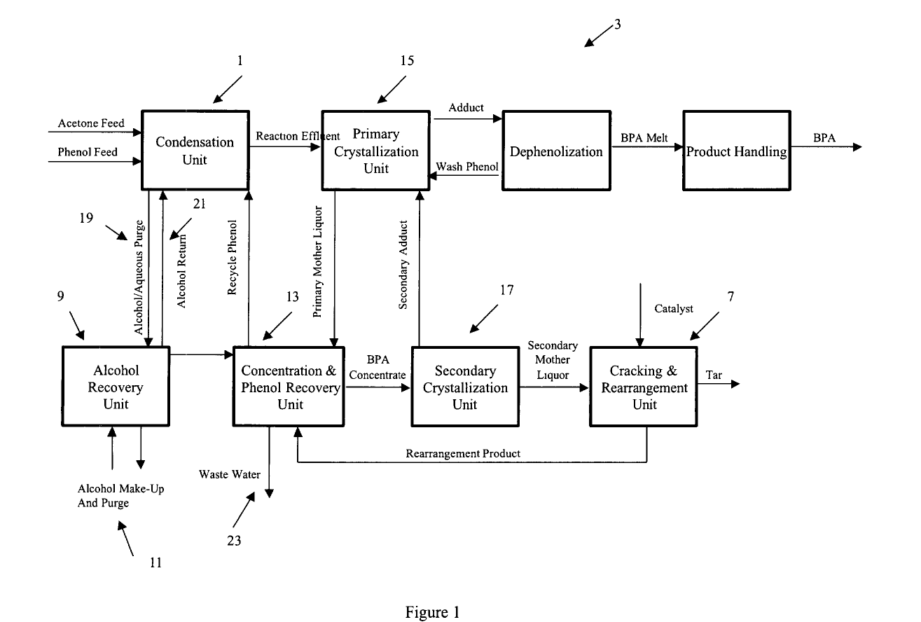

The invention can be adapted for use with any conventional acetone-phenol process for producing BPA. For example, FIG. 1 is a block diagram of such a conventional process, modified according to the preferred embodiment, wherein a lower alcohol is present in a condensation unit 1.

The reaction can be carried out in a temperature range between about 55° C. and about 120° C. At the lower temperatures the reaction rate is so slow as to be impractical, whereas the upper limit is established as the maximum recommended temperature for a cation resin catalyst in order to achieve acceptable catalyst life. A range of between about 80° C. and about 120° C. is preferred.

As used herein, the term “lower alcohol” is intended to mean any branched or unbranched alcohol having from 1 to 4 carbon atoms, i.e., C1 to C4 alcohols, or isomers thereof, including mixtures thereof. Thus, a lower alcohol(s) used in accordance with the invention can be any such alcohol(s) which exhibits similar behaviour for the purposes described herein. Examples of such alcohols are primary alcohols; for example, methanol, ethanol, n-propanol and n-butanol; and branched alcohols: for example, iso-propanol, sec-butanol, tertiary-butanol and iso-butanol.

The addition of a lower alcohol to the BPA production process can suppress to a large extent the production of by-products, and have a favourable effect on selectivity for the para, para-isomer. In a commercial context this has a significant cost benefit. The selectivity is at least sufficiently enhanced so as to reduce downstream processing. Such processing can include, for example, isomerization and cracking carried out in cracking and rearrangement unit 7. For example, there would be significantly less tar to be disposed of, either by incineration or landfill.

Most modem BPA production processes are based on a heterogeneous strong acid catalyst, and the process of the preferred embodiment also uses such catalysts. Examples of such catalysts are styrene-divinyl benzene sulphonated resins, super acid catalysts such as Nafion® (DuPont), perfluorsulphonic acid polymers, and novel catalysts where strong acids such as hydrochloric acid are immobilized on a carrier. Some of these catalysts are more robust than the styrene-divinyl benzene resins, and it would be expected that these would permit a higher operating temperature.

In accordance with the invention, the lower alcohol is not consumed in the production process. For example, in FIG. 1, the alcohol is separated from the aqueous stream from the condensation unit 1 by means of simple distillation in an alcohol recovery unit 9. As will be appreciated by those skilled in the art, an operation of this nature would require some small bleed and make-up of alcohol, e.g., 11 in FIG. 1, to control the build up of impurities in this stream.

To better understand the invention, it is helpful to further understand a conventional acetone-phenol BPA production process.

BPA is conventionally produced in a process similar to that shown in FIG. 1, except the conventional process lacks the alcohol recovery unit 9. This unit 9 is for the addition and recovery of alcohol as part of the process described herein. In the conventional process, the aqueous purge is directed to the concentration and phenol recovery unit 13. The conventional process operates by the condensation of two moles of phenol with one mole of acetone over a strong acid catalyst in the condensation unit 1. The chemistry is as follows:

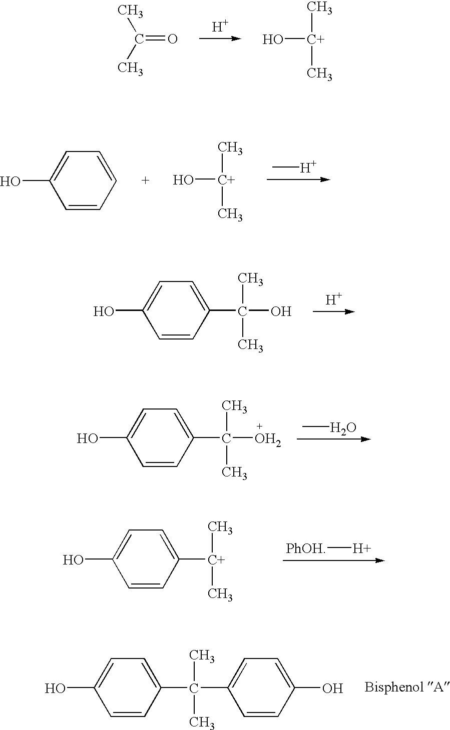

In addition to the desired product, para, para-isomer (BPA), there is always some ortho, para-isomer formed which must be eliminated from the final product. Acetone, under these reaction conditions, can follow an alternate reaction pathway via aldol condensation which results in the production of, among other by-products, mesityl oxide. Mesityl oxide is a very reactive compound that can combine with phenol and BPA itself to make a large number of refractory by-products.

The reaction product is separated and the BPA is purified by fractional crystallization; in FIG. 1 at the primary crystallization unit 15 and secondary crystallization unit 17. The unwanted ortho, para-isomer is isomerized in the cracking and rearrangement unit 7 as shown in FIG. 1 (at a cost) to yield more of the desired para, para-isomer. Unreacted acetone and phenol are separated for recycle.

The water produced in the reaction is rejected and the tar fraction, the refractory by-products, is cracked in the cracking and rearrangement unit 7 to revert some of these compounds to phenol and acetone for recycle. A large part of the by-products however must be rejected as tar.

Originally, the conventional process was carried out in batch with a strong mineral homogeneous acid catalyst such as sulphuric or hydrochloric acid. Use of homogeneous catalysts required a neutralization step and a distillation step. The free mineral acids caused corrosion problems resulting in a significant amount of down time. Nevertheless, the invention is applicable to such a process.

Current conventional processes are usually continuous and they utilize, for the most part, strong acid ion exchange resins. These solid acids avoid the neutralization step; however, because they are based on a styrene-divinyl benzene structure they are limited to about a 120° C. operating temperature in order to achieve reasonable catalyst life. Catalyst life is a known concern in acetone-phenol BPA processes, for example U.S. Pat. No. 5,777,180 to June et al issued Jul. 7, 1998 attempts to address that concern.

As shown by the relatively minor modifications required to a conventional process, a modified process 3 required to implement the principles described herein is straight forward. The lower alcohol, such as methanol, is introduced into the condensation unit I where the acetone-phenol reaction takes place. Wastewater and lower alcohol are removed as bottom product from the condensation unit 1 at the alcohol/acqueous purge 19. The alcohol is then separated in the alcohol recovery unit 9. This can be accomplished via a simple distillation, with the alcohol overhead product being recycled to the condensation unit 1 at alcohol return 21. There should be little or no further plant modifications required to take advantage of this proposed improved BPA process 3. Wastewater is then further separated by the concentration and phenol recovery unit 13 and removed at 23.

### EXAMPLE 1

Modified Process Trials using Methanol

Experiments designed by the inventor have been performed to demonstrate empirically that the addition of methanol to an acetone-phenol BPA production process significantly improves the selectivity of the process. Refractory by-products are suppressed and there is an improvement in the para, para-/ortho, para-isomer ratio. Methanol is not consumed in the process 3, rather it is substantially recycled, thus not contributing significantly to the overall cost of BPA production.

Sample experiments using methanol in the reaction vessel are set out in detail below. The experiments were performed in small batches and not optimized for commercial production. In order to view relative results, the experiments were also performed using DMP as a replacement for acetone in the production of BPA. From the experiments referred to above, it is known that DMP produced an increase in desirable isomer selectivity when measured against traditional acetone-phenol BPA production processes, while the amount of undesired by-products did not seem to be significantly affected.

It was found that the methanol-acetone-phenol process not only preserved the isomer selectivity improvement achieved with substituting DMP for acetone, but also that by-product production was greatly reduced.

1. Measure the proper volumes of methanol and acetone, as well as the proper mass of phenol and put in a three-neck round-bottomed flask. The precise measurements can be found in Table 1.

2. Add 2.5 g of Amberlyst®-15 (acid cation resin) to the flask.

3. Add a magnetic stir bar to the flask.

4. Connect a reflux condenser to the flask. Cover the other two necks of the flask with a stopper and parafilm.

5. Turn on the water flow to the reflux condenser and fill the water bath reserve with water.

6. Heat to 70° C. or desired temperature.

7 Immerse the flask in the water bath.

8. Turn the magnetic stir plate on.

TABLE 1
Concentration of Reactants

| Trial (Mol Ratios) | Phenol (g) | Acetone (ml) | Methanol (ml) |
|---|---|---|---|
| 1:2:2 | 31.18 | 48.13 | 26.87 |
| 3:2:2 | 62.35 | 32.08 | 17.92 |
| 2:1:1 | 66.51 | 25.66 | 14.34 |
| 5:2:2 | 62.36 | 25.67 | 14.34 |
| 3:1:1 | 99.77 | 25.70 | 14.34 |
| 7:2:2 | 87.31 | 19.25 | 10.75 |
| 4:1:1 | 99.97 | 19.25 | 10.75 |

Control Trial

1. Add 75 ml of 2,2-dimethyoxypropane to 28.46 g of phenol in a three-neck round-bottomed flask.

2. Repeat steps 2-8 of modified process.

Results

Samples taken in the trials were analyzed by gas chromatography with a mass spectroscopy detector (GC-MS). The GC-MS results were compared against a known standard for BPA to determine the quantity of BPA produced.

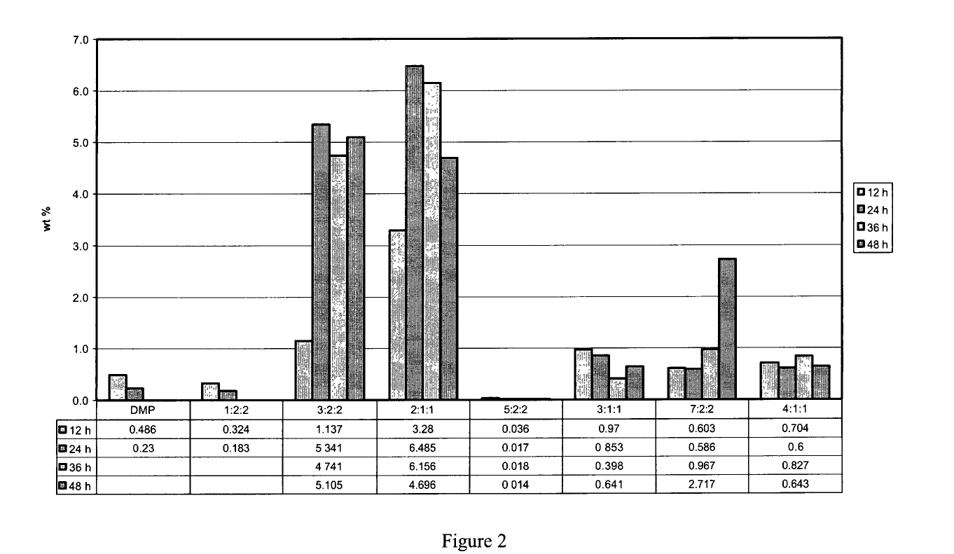

The resulting yield data is shown in FIG. 2. While the yields are low by industrial standards, it is important to note that the experiments were conducted batch-wise with limited time. Qualitatively the results serve the purpose for identifying most preferred mole ratios of reactants. It will be appreciated by those skilled in the art that the process can be optimized to achieve an acceptable yield in a commercial application.

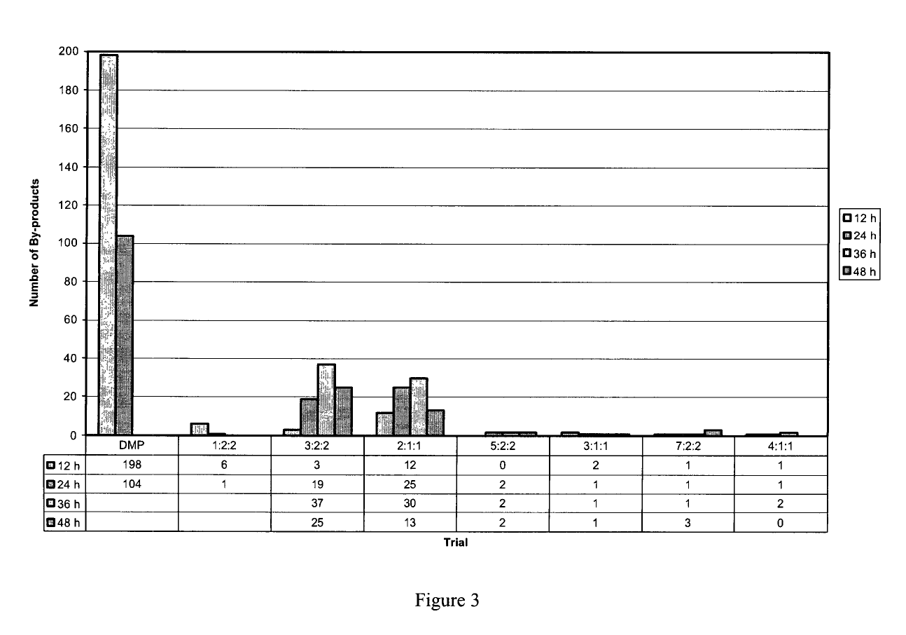

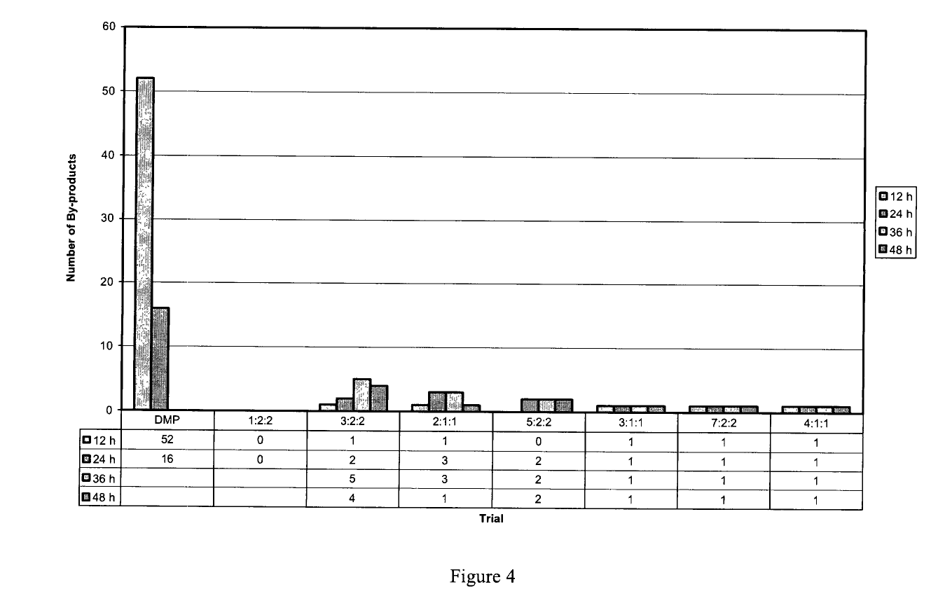

The GC-MS data also indicate a reduction in by-product formation. This is pictorially represented in FIGS. 3 and 4. FIG. 3 indicates the total number of by-products in the control (DMP) process versus the modified process. The DMP trial results are consistent with the results from the conventional acetone-phenol process. The results from the modified process indicate a significant reduction in number of by-products from nearly 200 to less than 40. Also significant is the reduction in by-products similar in structure to BPA. As indicated in FIG. 4, this number drops from over 50 to under 5.

These results confirm reduction in by-product formation achieved with the modified process.

### EXAMPLE 2

Modified Process Trials Using Other Primary Alcohols

Example 1 indicated the utility of adding methanol, a primary lower alcohol, to the BPA process to enhance reaction selectivity for BPA. Further experiments were conducted to compare methanol to other lower alcohols. The method of Example 1 was repeated using 10 g of Amberlyst-15 catalyst and a reaction temperature of 80EC. Table 2 details the molar ratios of the reactants, and the results rovided in Table 3.

TABLE 2
Molar Ratios of Reactants

| | | | | 2,2- | | |
|---|---|---|---|---|---|---|
| | | | Iso- | Dimethoxy | | |
| Trial | Methanol | Ethanol | Propanol | propane | Phenol | Acetone |
| 1 | 1 | — | — | 1 | 1 | |
| 2 | 1 | — | — | — | 1 | 1 |
| 3 | — | 1 | — | — | 1 | 1 |
| 4 | — | 1 | — | 1 | 1 | — |
| 5 | 0.75 | — | — | — | 1 | 1 |
| 6 | 1.25 | — | — | — | 1 | 1 |
| 7 | 1.25 | — | — | 1 | 1 | — |
| 8 | — | — | 1 | — | 1 | 1 |
| 9 | — | — | 1 | 1 | 1 | — |

TABLE 3
Results

| | | | | % Yield | Ratio |
|---|---|---|---|---|---|
| | | BPA | % Yield | of Side | BPA:Side |
| Trial | Description | (ppm) | of BPA | Reactions | Reactions |
| 1 | Methanol & DMP | 4961.4 | 22.8 | 29.36 | 0.8 |
| 2 | Methanol & Acetone | 5466.4 | 17.5 | 55.3 | 0.3 |
| 3 | Ethanol & Acetone | 3546.7 | 31.7 | 12.6 | 2.5 |
| 4 | Ethanol & DMP | 181.7 | 2.4 | 0.5 | 4.8 |
| 5 | Methanol & Acetone | 413.8 | 3.8 | 63.5 | 0.1 |
| 6 | Methanol & Acetone | 1524.2 | 36.6 | 6.4 | 5.7 |
| 7 | Methanol & DMP | 412.9 | 12.3 | 27.8 | 0.4 |
| 8 | Propanol & Acetone | 2645.7 | 22.6 | 32.6 | 0.7 |
| 9 | Propanol & DMP | 186.7 | 2.1 | 30.4 | 0.1 |

Similar to Example 1, there was a high degree of variability in the results, compared to industrial standards. This can be explained by the fact that the experiments were conducted batchwise with limited time. The results nevertheless indicate that BPA was produced with all of the alcohols used. It will be appreciated by those of ordinary skill in the art that under more controlled conditions the process can be optimized to decrease variability in the results and improve yields.

It will be understood by those skilled in the art that this description is made with reference to the preferred embodiment and that it is possible to make other embodiments employing the principles of the invention which fall within its spirit and scope as defined by the following claims.

I claim:

1. A method of making BPA comprising
reacting acetone and phenol over a strong acid catalyst in the presence of one or more lower alcohols selected from methanol, ethanol, n-propanol, iso-propanol, n-butanol, sec-butanol, tertiary-butanol and iso-butanol.

2. The method of claim 1, wherein at least one mole of lower alcohol is present for each mole of acetone.

3. The method of claim 1, wherein at least two moles of lower alcohol are present for each mole of acetone.

4. The method of claim 3, wherein there is no more than four moles of lower alcohol for each mole of acetone.

5. The method of claim 3, wherein at least one mole of phenol is present for each mole of acetone prior to reacting the acetone and phenol.

6. The method of claim 3, wherein the lower alcohol is selected from the group consisting of methanol, ethanol, propanol, and butanol.

7. The method of claim 3, wherein the lower alcohol is isopropyl alcohol.

8. The method of claim 3, wherein the lower alcohol is tertiary butyl alcohol.

9. The method of claim 3, wherein the lower alcohol is at least one of methanol, ethanol, propanol and butanol.

10. The method of claim 3, wherein the lower alcohol is methanol.

11. The method of claim 3, wherein the lower alcohol is ethanol.

12. The method of claim 3, wherein the lower alcohol is propanol.

13. The method of claim 3, wherein the lower alcohol is butanol.

14. The method of claim 3, wherein the acetone and phenol are reacted at less than about 120° C.

15. The method of claim 3, wherein the acetone and phenol are reacted at greater than about 55° C.

16. The method of claim 3, wherein the acetone and phenol are reacted at greater than about 70° C.

17. The method of claim 3, wherein the catalyst is an acid cationic resin.

18. A method of making BPA comprising:
reacting acetone and phenol over a strong acid catalyst in the presence of methanol, wherein at least one mole of methanol is present for each mole of acetone.

19. A method of making BPA comprising:
adding at least one lower alcohol selected from methanol, ethanol, n-propanol, iso-propanol, n-butanol, sec-butanol, tertiary-butanol and iso-butanol to a condensation unit of an acetone-phenol BPA production process, wherein at least one mole of the lower alcohol is present for each mole of acetone in the condensation unit.
